(19) United States
(12) Patent Application Publication
(10) Pub. No.: US 2007/0033742 A1
(43) Pub. Date: Feb. 15, 2007

(54) Pyrazole azomethines and colorants containing these compounds

(76) Inventor: Otto Gottel, Marly (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/291,665

(22) Filed: Dec. 1, 2005

(30) Foreign Application Priority Data
Dec. 2, 2004 (DE) 102004058059.6

(51) Int. Cl. A61K 8/00

(52) U.S. Cl. 8/405

(57) ABSTRACT

The object of the present invention is new pyrazole azomethines of Formula (I), (Ia), or (Ib), 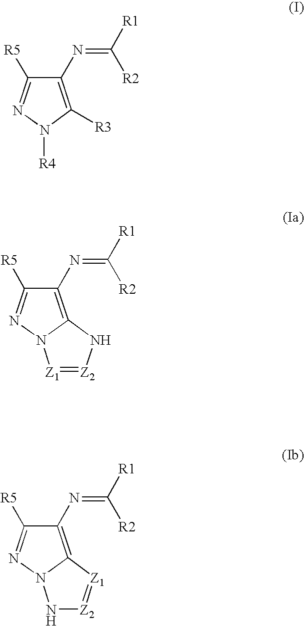 as well as agents containing these compounds for oxidative coloring of keratin fibers.

The present invention concerns new pyrazole azomethines as well as agents containing these compounds to color keratin fibers such as, for example, hair, silk, or wool.

The process of coloring hair is currently subjected to the most varied of trends. In the past, hair was colored primarily to cover gray portions, while today, there is increasingly more demand for integrating the hair color into the current fashion trends as well as for making it an expression of personality.

As in the past, there are currently two established methods available for coloring hair. One method is oxidative hair coloring, which leads to very permanent coloring results. The other method is tinting, which normally has less permanent results.

Oxidative hair coloring systems use developers and couplers that result in a broad color spectrum. The dye precursors that are documented in numerous patent texts are primarily electron-rich benzoic-aromatic or even heteroaromatic compounds. Due to the fact that these compounds are sensitive to oxidation as a result of being electron-rich, compounds that have alkaline groups are particularly used as acidic adducts. The acidic adducts have the advantage in that they are largely protected against air oxidation due to protonation of the amino functions; however, with the formulation of these types of raw materials into hair colorants, the pH value is normally adjusted to be alkaline by adding ammonia or ethanolamine. This results in salt being formed from the acidic adducts; these salts stress cosmetic formulations significantly. The higher salt content can cause phase separations in the worst case scenario; in addition, crystalline depositions can be observed with fluid formulations due to the higher alcohol content. The result of this is that various nuances requiring active color components cannot be marketed because of the reasons mentioned.

To prevent oxidative processes in the colorant precursors on the one hand and to protect cosmetic compositions from increased salt content on the other, the object was thus to provide colorant precursors that are adequately protected against air oxidation and also do not cause any salt load in the formulations. Furthermore, the new dyes should not have any negative effect on the color capacity of the formulations. Furthermore, it is intended to enable the production of ionic compounds, which, even as organic salts, hardly stress the formulations.

The use of nonionic azomethines in cosmetic compositions is already known from DE-OS 35 45 245, wherein the compounds described therein are used as direct-penetrating dyes. The use of iminophenoles to bleach skin pigments is described in WO 99/22707.

Surprisingly, it was then determined that protected compounds of Formula (I), in which activating amino groups are derived, are adequately stable with respect to air oxidation without influencing their activity in peroxide-containing colorants.

The object of the present application is thus pyrazole azomethines of Formula (I), 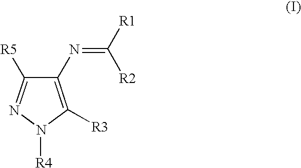
wherein
R1 and R2, independently from one another, represent hydrogen, a dimethylamino group, an aliphatic, aromatic, or heteroaromatic group, which, if necessary, is substituted with one or more amino groups, hydroxyl groups, nitrile groups, alkoxy groups, alkylsulfonyl groups, or keto groups, or groups R1 and R2, together with the carbon atom on which they sit, form cyclic or heterocyclic, saturated or unsaturated compounds, wherein R1 or R2 can additionally contain an ionic grouping, for example, a carboxylic acid group, a sulfonic acid group, a quaternary ammonium group, a pyridinium group, or an imidazolium group;
R3 can be a hydroxyl group, an amino group, a straight-chain or branched C1-C5 alkylamino group, a hydroxyethylamino group, a 2,3-dihydroxy propylamino group, or a benzylamino group, wherein the amino group is preferred;
R4 represents hydrogen, a straight-chain or branched C1-C12 alkyl group, a straight-chain or branched C2-C5 mono- or polyhydroxyalkyl group, a straight-chain or branched C3-C6 alkoxyalkyl group, a phenyl group, or a benzyl group, which, if necessary, are substituted with one or more C1-C3 alkyl groups, chlorine atoms, bromine atoms, trifluoromethyl groups, C1-C3 alkoxy groups, hydroxyethoxy groups, nitrile groups, hydroxyl groups, or is either a five-membered or six-membered heteroaromatic group, for example, a pyridine group, a pyrimidine group, a pyridazinyl group, a triazinyl group, a furyl group, a thienyl group, a pyrrolyl group, an imidazolyl group, a pyrazolyl group, a triazolyl group, which, if necessary, are substituted with one or more C1-C3 alkyl groups, fluorine atoms, chlorine atoms, bromine atoms, trifluoromethyl groups, methoxy groups, methanesulfonyl groups, carboxylic acid groups, carboxylic acid methylester groups, or carboxylic acid ethylester groups, phenyl groups, or hydroxymethyl groups, wherein annellated compounds such as, for example, quinoline groups, isoquinoline groups, or quinoxaline groups can be considered as the heteromatic group; and
R5 represents hydrogen, a straight-chain or branched C1-C6 alkyl group, a C1-C4 hydroxyalkyl group, a C1-C4 aminoalkyl group, a C1-C8 alkylamino group, a di(C1-C8)alkylamino group, a
C1-C4 alkylamino(C1-C4)alkyl group, a di(C1-C4)alkylamino(C1-C4)alkyl group, an aryl group, a heteroaryl group, a carboxylic acid group, a carboxylic acid methylester group, a carboxylic amide group, or a nitrile group, wherein hydrogen is preferred;
or R3 and R4 are bridged with one another so that a compound of Formula (Ia) or (Ib) is formed, 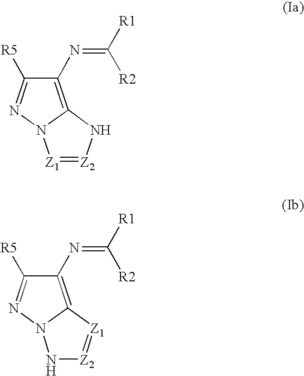
wherein
Z1 and Z2, independently from one another, stand for a nitrogen atom or a carbon atom substituted with a hydrogen atom, a straight-chain or branched C1-C5 alkyl group, a benzyl residue, a trifluoromethyl group, a hydroxymethyl group, a hydroxyethyl group, a methoxy group, or a phenyl group substituted with a methyl group, a chlorine atom, a hydroxy group, a methoxy group, or a trifluoromethyl group, and residue groups R1, R2, and R5 have the aforementioned meaning.

Preferred compounds of Formula (Ia) and (Ib) are such compounds, in which Z1 and Z2, independently from one another, stand for a nitrogen atom, a CH group or a C—CH₃ group, wherein those compounds of Formula (Ia) and (Ib) are especially preferred, in which Z1 represents a C—CH₃ group and Z2 represents a CH group, or Z2 represents a C—CH₃ group and Z1 represents a CH group.

The azomethines can be manufactured using customary synthesis methods; an overview of the common synthesis methods can be found, for example, in “Houben-Weyl,” Methoden der organischen Chemie [Methods of Organic Chemistry], Vol. E14b, 222-281, Georg-Thieme-Verlag [publishing company], Stuttgart (1990). The azomethines of the present invention are preferably manufactured from pyrazoles with at least one unsubstituted amino group (II) and the aldehydes and/or ketones of Formula (III). In individual cases, it can be advantageous to carry out the synthesis via the condensation reaction of nitroso compounds (IV) with methylene-active compounds (V).

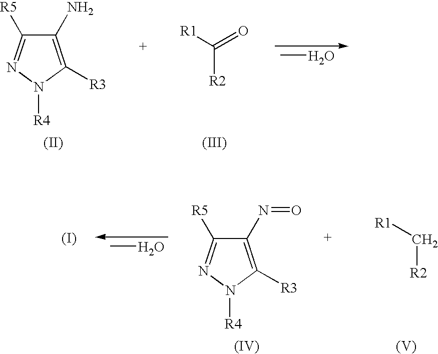

Examples of suitable pyrazoles (II) can be, for example: 4,5-diamino-1-methylpyrazole, 4,5-diamino-1-isopropyl pyrazole, 4,5-diamino-1-n-pentyl pyrazole, 4,5-diamino-1-(2-hydroxyethyl)pyrazole, 4,5-diamino-1-(2-methoxyethyl)pyrazole, 4,5-diamino-1-phenyl pyrazole, 4,5-diamino-1-(4-methylphenyl)pyrazole, 4,5-diamino-1-(4-methoxyphenyl)pyrazole, 4,5-diamino-1-(2-pyridinyl)pyrazole, 4,5-diamino-1-(4-methoxybenzyl)pyrazole, 3-[(4,5-diamino-1H-pyrazol-1-yl)methyl]-1-methylpyridinium chloride, 4,5-diamino-1,3-dimethyl-1H-pyrazole, 4,5-diamino-1-(2-hydroxyethyl)-3-methyl-1H-pyrazole, 4,5-diamino-1-methyl-3-phenyl-1H-pyrazole, 4,5-diamino-1-(2-hydroxyethyl)-3-phenyl-1H-pyrazole, 4,5-diamino-3-methyl-1-phenyl pyrazole, 4-[(4,5-diamino-1-isopropyl-1H-pyrazol-3-ylmethyl)amino]phenol, 2-isopropyl-5-morpholin-4-ylmethyl-2H-pyrazol-3,4-diamine, 2-isopropyl-5-thiophen-2-yl-2H-pyrazol-3,4-diamine, and 4-amino-1,3-dimethyl-5-ethylamino-1H-pyrazole, as well as acidic adducts thereof.

Carbonyl components (III) suitable for deriving the pyrazoles (II) of Formula (II) can be, for example, aliphatic, benzoaromatic, or heterocyclic five-membered or six-membered aldehydes, wherein the following aromatic aldehydes are preferred: benzaldehyde, anisaldehyde, pyridine-2-carbaldehyde, pyridine-3-carbaldehyde, pyridine-4-carbaldehyde, 2-formyl-1-methylpyridinium methylsulphate, 3-formyl-1-methylpyridinium methylsulphate, 4-formyl-1-methylpyridinium methylsulphate, furan-2-carbaldehyde, thiophene-2-carbaldehyde, vanillin, isovanillin, 4-dimethylamino benzaldehyde, 4-hydroxy benzaldehyde, as well as bifunctional aldehydes such as phthaldialdehyde, isophthalaldehyde, or terephtaldehyde; monoaldehydes are especially preferred.

In special cases, aldehyde derivatives can be used instead of the free aldehydes, such as their hydrates, aldehyde sulfite adducts, imides, aniles, thioaldehydes, half-acetals, or acetals thereof.

The following aldehyde derivatives should be mentioned in particular: 2,2-dimethoxy ethanol and 2,2-dimethoxy ethylamine.

Dimethylformamide acetals that lead to imido formamides are also suitable as dimethylformamide derivatives.

The following can be mentioned as ketones of the Formula (III): acetone, trifluoroacetone, hexafluoroacetone, hydroxyacetone, dihydroxyacetone, 1,3-diamino acetone, dimethylamino acetone, 3-chloro-2-butanone, phenacylamine, acetoin, cyclopropyl methylketone, cyclohexanone, acetophenone, 2,4-dihydroxyacetophenone, benzalacetone, 3-acetyl indole, 2-acetyl pyrrole, 4-chromanone, 2-indanone, mesityl oxide, trans-4-methoxy-3-butylene-2-on(1-1-3), 1-methyl-4-piperidone, 2-octadecanone, 3-methylthio-2-butanone, 3-pentene-2-on, phoron, pseudoionone, γ-pyrone, pivaloyl acetonitrile, 2,2,6,6-tetramethyl-4-piperidone, and tropolone.

The following can be mentioned, for example, as suitable nitroso compounds of Formula (IV): 5-amino-1-methyl-4-nitroso pyrazole, 5-amino-1-isopropyl-4-nitroso pyrazole, 5-amino-4-nitroso-1-n-pentyl pyrazole, 5-amino-1-(2-hydroxyethyl)-4-nitroso pyrazole, 5-amino-1-(2-methoxyethyl)-4-nitroso pyrazole, 5-amino-4-nitroso-1-phenyl pyrazole, 5-amino-1-(4-methylphenyl)-4-nitroso pyrazole, 5-amino-1-(4-methoxyphenyl)-4-nitroso pyrazole, 5-amino-4-nitroso-1-(2-pyridinyl)pyrazole, 5-amino-1-(4-methoxybenzyl)-4-nitroso pyrazole, 5-amino-1,3-dimethyl-4-nitroso-1H-pyrazole, 5-amino-1-(2-hydroxyethyl)-3-methyl-4-nitroso-1H-pyrazole, 5-amino-1-methyl-4-nitroso-3-phenyl-1H-pyrazole, 5-amino-1-(2-hydroxyethyl)-4-nitroso-3-phenyl-1H-pyrazole, and 5-amino-3-methyl-4-nitroso-1-phenyl pyrazole.

Open-chain as well as cyclic compounds (R1 bound to R2) are suitable as methylene-active compounds that are a possibility for realizing nitroso compounds of Formula (IV), wherein, for example, acetylacetone, malodinitrile, benzoylacetonitrile, cyanoacetic acid ethyl ester, cyanoacetamide, cyclopentane-1,3-dione, cyclohexane-1,3-dione, indane-1,3-dione, Meldrum's acid, barbituric acid, 3-methyl-1-phenyl pyrazolone, and 1-ethyl-1,2-dihydro-6-hydroxy-4-methyl-2-oxopyridine-3-carbonitrile can be mentioned as methylene-active compounds.

Due to the multitude of commercially available aldehydes and ketones, it is possible to adapt the carbonyl components to the amine components in such a manner so as to obtain optimum performance in various cosmetic compositions with the resulting azomethines.

The following compounds of the Formula (I) are particularly preferred: 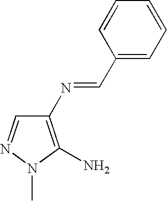
1-methyl-N⁴-[phenylmethylidene]-1H-pyrazole-4,5-diamine, 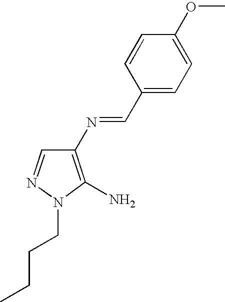
N⁴-{[4-(methyloxy)phenyl]methylidene}-1-pentyl-1H-pyrazole-4,5-diamine, 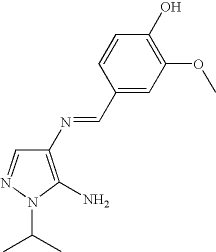
4-({[5-amino-1-(1-methylethyl)-1H-pyrazol-4-yl]imino}methyl)-2-(methyloxy)phenol, 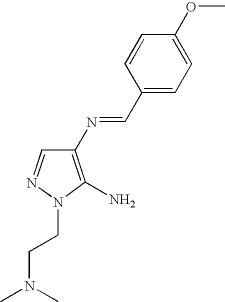
1-[2-(dimethylamino)ethyl]-N⁴-[(4-methoxyphenyl)methylidene]-1H-pyrazole-4,5-diamine, 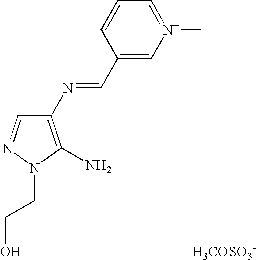
3-({[5amino-1-(2-hydroxyethyl)-1H-pyrazol-4-yl]imino}methyl)-1-methyl pyridinium methylsulphate, 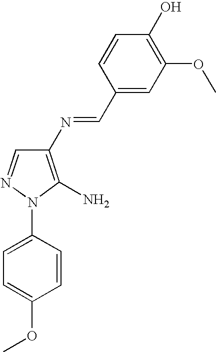
4-({[5-amino-1-(4-methoxyphenyl)-1H-pyrazol-4-yl]imino}methyl)-2-methoxyphenol, 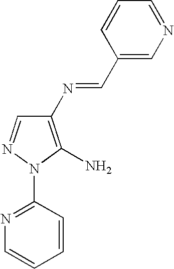
1-(2-pyridinyl)-N⁴-[3-pyridinylmethylidene]-1H-pyrazole-4,5-diamine, 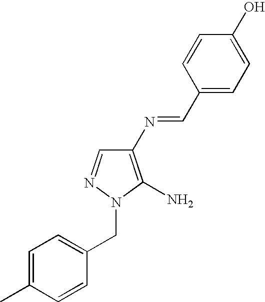
4-({[5-amino-1-(4-methylbenzyl)-1H-pyrazol-4-yl]imino}methyl)phenol, and 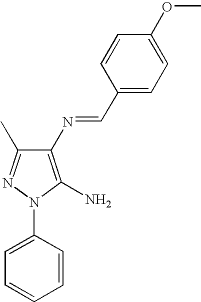
3-methyl-N4-{[4-(methyloxy)phenyl]methylidene}-1-phenyl-1H-pyrazole-4,5-diamine.

The compounds of Formula (I) according to the present invention sometimes enable significantly more color intensities than the corresponding non-derived basic bodies. They are therefore very well suited as dye precursors for coloring keratin fibers in the oxidative system. Although the compounds of the Formula (I) are particularly well suited for use to color keratin fibers, for example, wool, silk, or hair (particularly human hair), it is, in principle, also possible to color other natural or synthetic fibers, for example, cotton or Nylon 66, using these compounds.

For application in oxidative hair colorants, it is advantageous if the azomethines do not have any or only slight inherent color. Indoanilines, indophenols, and indamines are thus limited in their applicability due to their inherent color and generally are not considered to be part of the preferred compounds.

An additional object of the present invention is an agent for oxidative coloring of keratin fibers such as, for example, wool, fur, feathers, or hair, and particularly human hair, wherein it contains at least one compound of general Formula (I).

The azomethines of Formula (I) are contained in the colorant of the present invention in a quantity of about 0.005 to 20 percent by weight, wherein a quantity of about 0.01 to 10 percent by weight, and particularly 0.1 to 8 percent by weight, is preferred.

The compounds of Formula (I) can be used either alone or in conjunction with known developer substances and/or coupler substances typically used in oxidative color systems.

The following can be mentioned, in particular, as suitable coupler substances: N-(3-dimethylamino-phenyl)urea, 2,6-diamino pyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 2,4-diamino-1-(3-hydroxypropoxy)benzene, 2,4-diamino-1-(3-methoxypropoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di-(2-hydroxyethyl)amino]aniline, 4-amino-2-di-[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di-(2,4-diaminophenoxy)propane, di-(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 1,3-dihydroxy-2,4-dimethylbenzene, 3,4-methylenedioxyphenol, 3,4-methylenedioxyaniline, 5-[(2-hydroxyethyl)amino]-1,3-benzodioxol, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 4-hydroxyindole, 5-hydroxyindole, 6-hydroxyindole, 7-hydroxyindole, and 2,3-indoline dione, or salts thereof.

To create tinting that is very natural as well as fashionable red tinting, it is particularly advantageous to use compounds of Formula (I) in conjunction with additional developer substances. p-phenylenediamines, p-aminophenoles, and 4,5-diaminopyrazoles or salts thereof are possible developer substances.

The following developer substances should be mentioned in particular:

1,4-diaminobenzene (p-phenylenediamine), 1,4-diamino-2-methylbenzene (p-toluene diamine sulfate), 1,4-diamino-2,6-dimethylbenzene, 1,4-diamino-2,5-dimethylbenzene, 1,4-diamino-2,3-dimethylbenzene, 2-chloro-1,4-dimethylbenzene, 4-phenylaminoaniline, 4-dimethylaminoaniline, 4-diethylaminoaniline, 4-[di(2-hydroxyethyl)amino]aniline, 4-[(2-methoxyethyl)amino]aniline, 4-[(3-hydroxypropyl)amino]aniline, 1,4-diamino-2-(2-hydroxyethyl)benzene, 1,4-diamino-2-(1-hydroxyethyl)benzene, 1,4-diamino-2-(1-methylethyl)benzene, 1,3-bis[(4-aminophenyl)-(2-hydroxyethyl)amino]-2-propanol, 1,8-bis(2,5-diaminophenoxy)-3,6-dioxaoctane, 4-aminophenol, 4-amino-3-methylphenol, 4-methylaminophenol, 4-amino-2-(aminomethyl)phenol, 4-amino-2-[(2-hydroxyethyl)amino]methylphenol, 4-amino-2-(methoxymethyl)phenol, 4-amino-2-(2-hydroxyethyl)phenol, 5-amino salicylic acid, 2,5-diaminopyridine, 2,4,5,6-tetra amino pyrimidine, 2,5,6-triamino-4-(1H)-pyrimidone, 4,5-diamino-1-(2-hydroxyethyl)-1H-pyrazol, 4,5-diamino-1-(1-methylethyl)-1H-pyrazol, 4,5-diamino-1-(4-methylbenzyl)-1H-pyrazol, 1-(4-chlorobenzyl)-4,5-diamino-1H-pyrazol, 4,5-diamino-1-methyl-1H-pyrazol, 4,5-diamino-3-methyl-1-phenyl-1H-pyrazol, 4,5-diamino-1-methyl-1H-pyrazol, 4,5-diamino-1-pentyl-1H-pyrazol, 4,5-diamino-1-benzyl-1H-pyrazol, 4,5-diamino-1-(4-methoxybenzyl)-1H-pyrazol, 4,5-diamino-1-(2-hydroxyethyl)-3-methyl-1H-pyrazol, 2-aminophenol, 2-amino-6-methylphenol, and 2-amino-5-methylphenol, or salts thereof.

The aforementioned developer substances and coupler substances can each be used individually or in a mixture with each other, wherein the aforementioned known developer substances and coupler substances are each contained in the colorant of the present invention in a total quantity of about 0.01 to 20 percent by weight, with about 0.2 to 8 percent by weight being preferred.

Furthermore, the colorant of the present invention can also contain other color components, for example 4-(2,5-diamino benzylamino)aniline or 3-(2,5-diamino benzylamino)aniline, as well as other customary natural, nature-identical, or synthetic direct-penetrating dyes from the group of the anionic (acidic) and cationic (alkaline) dyes, the triarylmethane dyes, the nitro dyes, the dispersion dyes, and the azo dyes, for example, natural dyes such as indigo or henna, triphenyl methane dyes such as 4-[(4′-aminophenyl)-(4′imino-2″,5″-cyclohexadiene-1″-yliden)methyl]-2-methylamino benzene monohydrochloride (C.I. 42 510) and 4-[(4′-amino-3′-methylphenyl)-(4″-imino-3″-methyl-2″,5″cyclohexadiene-1″-yliden)methyl]-2-methylamino benzene monohydrochloride (C.I. 42 520), aromatic nitro dyes such as 4-(2′-hydroxyethyl)amino nitrotoluene, 2-amino-4,6-dinitrophenol, 2-amino-5-(2′-hydroxyethyl)amino nitrobenzene, 2-chloro-6-(ethylamino)-4-nitrophenol, 4-chloro-N-(2-hydroxyethyl)-2-nitroaniline, 5-chloro-2-hydroxy-4-nitroaniline, 2-amino-4-chloro-6-nitrophenol, 2-amino-6-chloro-4-nitrophenol, 2-chloro-6-(ethylamino)-4-nitrophenol, and 1-[(2′-ureidoethyl)amino-4-nitrobenzene, azo dyes such as 6-[(4′-aminophenyl)-azo]-5-hydroxynaphthalene-1-sulfonic acid sodium salt (C.I. 14 805), and dispersion dyes such as 1,4-diaminoanthraquinone and 1,4,5,8-tetraaminoanthraquinone.

The total concentration of direct-penetrating dyes in the agent of the present invention is about 0.01 to 10 percent by weight, with 0.1 to 5 percent by weight being preferred.

The herein before described combinations of the Formula (I) of the present invention with oxidative hair color precursors and/or direct-penetrating dyes are applied for coloring in a suitable color vehicle.

Moreover, the colorants can contain still other conventional additives, for example antioxidants such as ascorbic acid, mercaptoacetic acid or sodium sulfite, as well as perfume oils, penetrating agents, buffer systems, complexing agents, preservatives, wetting agents, emulsifiers, thickeners and conditioning agents.

The formulation of the colorant according to the present invention can be, for example, a solution, in particular an aqueous or aqueous alcoholic solution, a paste, a cream, a gel, an emulsion, or an aerosol preparation. The composition of the solution represents a mixture of dye components with the conventional additives for such formulations. It is also possible for the colorant of the present invention to be in the form of pellets, granulates, or powders, which are dissolved in an aqueous preparation—for example, in water or an aqueous oxidizing agent preparation.

Conventional additives in solutions, cremes, emulsions, or gels are, for example, solvents such as water, lower aliphatic alcohols, for example, ethanol, propanol or isopropanol, glycerin or glycols such as 1,2-propyleneglycol, and furthermore, wetting agents or emulsifiers from the anionic, cationic, amphoteric or nonionic classes of surface-active substances such as, for example, fatty alcohol sulfates, oxyethylated fatty alcohol sulfates, alkyl sulfonates, alkylbenzene sulfonates, alkyltrimethylammonium salts, alkyl betaines, oxyethylated fatty alcohols, oxyethylated nonylphenols, fatty acid alkanolamides and oxyethylated fatty acid esters, and moreover, thickeners such as higher fatty alcohols, starches, cellulose derivatives, petroleum jelly, paraffin oil and fatty acids, and conditioning agents such as cationic resins, lanolin derivatives, cholesterol, pantothenic acid, and betaine. The above-mentioned components are used in the conventional amounts for such purposes, for example, wetting agents and emulsifiers in concentrations of from about 0.5 to 30 percent by weight, thickeners in an amount of from about 0.1 to 30 percent by weight, and conditioning agents in a concentration of from about 0.1 to 5 percent by weight.

Depending on the composition, the dye of the present invention can be weakly acidic, neutral, or alkaline. In particular, it has a pH value of from 6 to 11.5, wherein the alkaline adjustment preferably occurs using ammonia or organic amines, for example, monoethanolamine and triethanolamine, or amino acids or inorganic bases such as sodium hydroxide and potassium hydroxide. It is also possible to use combinations of the aforementioned compounds, in particular, a combination of ammonium and monoethanolamine. For pH-adjustments to make the composition more acidic, the use of inorganic or organic acids, for example, phosphoric acid, acetic acid, citric acid or tartaric acid, can be considered.

For use for oxidative coloring of hair, the herein before described colorant (pH=6 to 11.5) is mixed with an oxidizing agent (pH=2 to 6.5) immediately before use. In this process, the pH value of the ready-to-use hair colorant adjusts itself to a pH value that is determined by the alkali quantity in the color vehicle and the acid quantity in the oxidizing agent as well as by the mixture ratio. Depending on the composition, the ready-to-use colorants can react weakly acidic, neutral, or alkaline and have a pH value of about 3 to 11, with 5 to 10 being preferred.

Hydrogen peroxide or addition compounds thereof with urea, melamine, sodium borate, or sodium carbonate in the form of a 3 to 12 percent, preferably 6 percent, aqueous solution, as well as atmospheric oxygen are possibilities for oxidizing agents to develop hair color. Based on a 6 percent content of free hydrogen peroxide in the oxidizing agent, the weight ratio between the hair colorant and the oxidizing agent is about 5:1 to 1:2, with, however, 1:1 being preferred. Larger quantities of oxidizing agents are primarily used with higher dye concentrations in the hair coloring agent or if a stronger bleaching of the hair is intended simultaneously.

A quantity of this mixture that is sufficient for the hair coloring treatment, generally about 60 g (2.1 oz) to 200 g (7.05 oz) depending on hair thickness, is applied to the hair, and the mixture is left on the hair for about 10 to 45 minutes, preferably 30 minutes, at 15° C. (59° F.) to 50° C. (122° F.); the hair is then rinsed with water and dried. If necessary, the hair is subsequently washed with shampoo and re-rinsed, as needed, with a weak organic acid such as citric acid or tartaric acid. Next the hair is dried.

The colorants of the present invention containing compounds of the Formula (I) enable hair coloring with extraordinary color fastness, particularly with regard to lightfastness, color fastness during washing, and fastness to rubbing. With respect to the color qualities, the hair colorants according to the present invention, depending on the type and composition of the color components, provide a wide range of various color shades, particularly in the area of fashionable red tones. The color tones distinguish themselves by their particular color intensity and luminous power. The extremely good color qualities of the colorants according to the present application are particularly demonstrated by the fact that these agents also enable uniform and lasting coloring even for hair that was previously damaged to varying degrees.

The following examples will illustrate the object of the invention without limiting the invention to these examples.

### EXAMPLES

### Example 1

### Producing 2-(5-amino-4-{[(4-methylphenyl)methylidene]amino}-1H-pyrazol-1-yl)ethanol

7.28 g (0.26 oz) (51.2 mmol) 4,5-diamino-1-(2-hydroxyethyl)pyrazole in 60 ml (2.0 oz) ethanol is added to 6.76 g (0.24 oz) (56.3 mmol) toluylaldehyde and agitated at room temperature. The solution obtained is initially red, from which a yellow product gradually precipitates. After 1 hour, suction is applied and the substance is washed with a little bit of cold ethanol. Drying in a vacuum at 40° C. (104° F.) results in 6.4 g (0.2 oz) (51% of theory) of pale yellow product.

¹H-NMR (DMSO-d₆): δ=8.47 (s, 1H); 7.72 (d, 2H); 7.55 (s, 1H); 7.24 (d, 2H); 5.36 (s broad, 2H); 4.94 (t, 1H); 3.95 (t, 2H); 3.67 (m, 2H); 2.34 ppm (s, 3H).

### Example 2

### Producing 2-(5-amino-4-{[3-pyridinylmethylidene]amino}-1H-pyrazol-1-yl)ethanol

The procedure is the same as in Example 1, wherein, instead of the toluylaldehyde, 6.03 g (0.21 oz) (56.3 mmol) pyridine-3-carbaldehyde is used.

Yield: 6.6 g (0.2 oz) (56% of theory) yellow product. ¹H-NMR (DMSO-d₆): δ=8.95 (d, 1H); 8.55 (m, signal superimposition, 2H); 8.18 (d, 1H); 7.59 (s, 1H); 7.43 (dd, 1H); 5.59 (s broad, 2H); 4.94 (t, 1H); 3.96 (t, 2H); 3.68 ppm (m, 2H).

### Example 3

### Producing 3-({[5amino-1-(2-hydroxyethyl)-1H-pyrazol-4-yl]imino}methyl)-1-methylpyridinium methylsulphate

2.31 g (0.08 oz) (10 mmol) of the substance from Example 2 is suspended in 40 ml (1.4 oz) acetonitrile and added to 1.39 g (0.05 oz) (11 mmol) dimethylsulfate and agitated 24 hours at room temperature, wherein the suspension changes color from yellow to orange. Suction is then applied and the substance is washed with a little bit of ethyl acetate. The substance is then dried in a vacuum at 40° C. (104° F.). This results in 3.2 g (0.1 oz) (89% of theory) of orange solid material.

¹H-NMR (DMSO-d₆): δ=9.36 (s, 1H); 8.84 (d, 1H); 8.76 (d, 1H); 8.50 (s, 1H); 8.12 (m, 1H); 7.64 (s, 1H); 5.98 (s, very broad, 2H); 4.37 (s, 3H); 3.97 (t, 2H); 3.69 (t, 2H); 3.38 (s, 3H).

### Example 4

### Producing 4-({[5-amino-1-(4-methylbenzyl)-1H-pyrazol-4-yl]imino}methyl)phenol

8.05 g (0.28 oz) (39.8 mmol) 4,5-diamino-1-(4-methylbenzyl)-1H-pyrazole is dissolved in 100 ml (3.38 oz) methanol/water 1:1 (v/v) and added to 5 g (0.2 oz) (41 mmol) 4-hydroxy benzaldehyde. The product precipitates after about 2 minutes. To complete the reaction, the product is further agitated for 20 more minutes at room temperature and then suctioned. The product is rinsed with water and dried in a vacuum at 40° C. (104° F.).

Yield: 10.3 g (0.36 oz) (84% of theory) yellow product. ¹H-NMR (DMSO-d₆): δ=9.79 (s broad, 1H); 8.40 (s, 1H); 7.65 (d, 2H); 7.53 (s, 1H); 7.12 (d, 2H); 7.07 (d, 2H); 6.81 (d, 2H); 5.44 (s broad, 2H); 5.10 (s, 2H); 2.27 ppm (s, 3H).

### Example 5

### Producing 3-methyl-N4-{[4-(methyloxy)phenyl]methylidene}-1-phenyl-1H-pyrazole-4,5-diamine

4 g (0.1 oz) (15.3 mmol) 4,5-diamino-3-methyl-1-phenyl pyrazole dihydrochloride and 0.5 g (0.02 oz) sodium sulfite are dissolved in 250 ml (8.45 oz) water, and the pH value is adjusted to 7 by adding a concentrated ammonia solution. Subsequently, 2.21 g (0.08 oz) (16.2 mmol) 4-methoxy benzaldehyde is added. After a few minutes, the reaction mixture becomes cloudy and an oily, orange-brown precipitate is formed. The mixture is diluted with 40 ml (1.4 oz) isopropanol and further agitated, wherein the oily deposition solidifies. After 2 hours agitation, suction is applied and the mixture is thoroughly washed with water. Drying in a vacuum at 40° C. (104° F.) results in 2.4 g (0.08 oz) (51% of theory) of yellowish solid material.

¹H-NMR (DMSO-d₆): δ=8.55 (s, 1H); 7.86 (d, 2H); 7.62 (m, 2H); 7.50 (m, 2H); 7.33 (m, 1H); 7.01 (d, 2H); 5.48 (s broad, 2H); 3.82 (s, 3H); 2.34 ppm (s, 3H).

### Example 6

### Producing N4-{[4-(methyloxy)phenyl]methylidene}-1-pentyl-1H-pyrazole-4,5-diamine

4 g (0.1 oz) (16.6 mmol) 4,5-diamino-1-pentyl pyrazole dihydrochloride and 0.5 g (0.02 oz) sodium sulfite are dissolved in 100 ml (3.38 oz) water and neutralized by adding a concentrated ammonia solution. 2.4 g (0.08 oz) (17.6 mmol) anisaldehyde in 40 ml (1.4 oz) isopropanol is added to this solution with the application of strong agitation. After a few minutes, the solution becomes cloudy and a yellowish precipitate is formed. After 2 hours, suction is applied and the solution is thoroughly washed with water. Drying in a vacuum at 40° C. (104° F.) results in 3.8 g (0.1 oz) (80% of theory) of yellow product.

¹H-NMR (DMSO-d₆): δ=8.43 (s, 1H); 7.76 (d, 2H); 7.50 (s, 1H); 6.99 (d, 2H); 5.38 (s, broad, 2H); 3.85 (m, signal superimposition, 5H); 1.66 (m, 2H); 1.27 (m, 4H); 0.86 ppm (t, 3H).

### Example 7

### Producing 4-({[5-amino-1-(1-methylethyl)-1H-pyrazol-4-yl]imino}methyl)-2-(methyloxy)phenol

4.76 g (0.18 oz) (20 mmol) 4,5-diamino isopropyl pyrazole sulfate and 0.5 g (0.02 oz) sodium sulfite are dissolved in 200 ml (6.76 oz) water, and the pH value is adjusted to 7 by adding a concentrated ammonia solution. Subsequently, 3.35 g (0.12 oz) (22 mmol) vanillin is added. After a few minutes, the reaction mixture becomes cloudy and light yellow crystals are formed. After 2 hours agitation, suction is applied and the mixture is thoroughly washed with water. Drying in a vacuum at 40° C. (104° F.) results in 4.27 g (0.15 oz) (78% of theory) of yellow solid material.

¹H-NMR (DMSO-d₆): δ=9.36 (s, broad, 1H); 8.37 (s, 1H); 7.50 (m, signal superimposition, 2H); 7.15 (dd, 1H); 6.80 (d, 1H); 5.32 (s, broad, 2H); 4.44 (septet, 1H); 3.84 (s, 3H); 1.30 ppm (d, 6H).

### Example 8

### Color Example

| | | |
|---|---|---|
| 0.30 | g (0.01 oz) | ascorbic acid |
| 0.40 | g (0.01 oz) | sodium sulfite |
| 10.00 | g (0.35 oz) | sodium lauryl ether sulphate, |
| | | 28% aqueous solution |
| 7.85 | g (0.28 oz) | ethanol |
| 12.5 | mmol | compound of Formula (I) according |
| | | to Table 1 |
| 12.5 | mmol | coupler substance according to |
| | | Table 1 |
| 9.10 | g (0.32 oz) | ammonia, 25% aqueous solution |
| balance to 100.00 | g (3.53 oz) | water, de-mineralized |

Immediately before use, 100 grams (3.53 oz) of the herein before color vehicle is mixed with 100 grams (3.53 oz) of a 6% aqueous hydrogen peroxide solution, and the ready-to-use dye solution obtained is applied to bleached hair in the required quantity. After an action period of 30 minutes at 40° C. (104° F.), the hair is washed with shampoo, rinsed with water, and dried. The color shades obtained and the L*a*b* values are compiled in Table 1.

The L*a*b* color measurements indicated in the present application were obtained using a color measurement device, the Chromameter II, manufactured by Minolta. The L* value stands for lightness (meaning the smaller the L* value, the greater the color intensity), while the a* value is a measure of the red portion (meaning the greater the a* value, the greater the red portion). The b* value is a measure of the blue portion of the color, wherein the blue portion becomes greater as the b* value becomes more negative.

[symbol]

TABLE 1
Color results from various developer/coupler combinations

| Color example | 8a | 8b | 8c | 8d | 8e |
|---|---|---|---|---|---|
| Dye precursors | resorcinol | m-aminophenol | 2-amino-5- | 2,4-diamino | 2-{[3-amino-4- |
| | | | methylphenol | phenoxy ethanol* | (methyloxy)phenyl]- |
| | | | | 2HCl | amino}ethanolsulfate |
| Dye according | Pale pink | Red | Orange-red | Red-violet | Dark red-violet |
| to Example 1 | L = 53.9; a = 31.2; | L = 37.0; a = 44.7; | L = 45.6; a = 49.1; | L = 24.4; a = 32.7; | L = 22.8; a = 26.6; |
| | b = 18.9 | b = 27.2 | b = 44.0 | b = 4.4 | b = 2.1 |
| Dye according | Pale pink | Dark red | Fiery red | Red-violet | Dark red-violet |
| to Example 2 | L = 55.7; a = 28.2; | L = 28.8; a = 41.9; | L = 38.8; a = 50.7; | L = 22.0; a = 27.2; | L = 20.6; a = 21.9; |
| | b = 17.1 | b = 20.5 | b = 38.0 | b = 3.1 | b = 1.2 |
| Dye according | Beige-orange | Blood-red | Orange-red | Red-violet | Dark red-violet |
| to Example 3 | L = 60.7; a = 22.6; | L = 34.9; a = 54.1; | L = 42.7; a = 49.5; | L = 25.0; a = 31.0; | L = 24.0; a = 22.6; |
| | b = 21.3 | b = 28.3 | b = 42.6 | b = 6.0 | b = 3.2 |
| Dye according | Dusky pink | Red | Orange-red | Red-violet | Dull violet |
| to Example 4 | L = 54.0; a = 22.2; | L = 31.2; a = 39.8; | L = 45.4; a = 42.5; | L = 24.0; a = 32.3; | L = 26.9; a = 27.0; |
| | b = 8.8 | b = 17.2 | b = 39.4 | b = 2.7 | b = −0.3 |
| Dye according | Flax colored | Light burgundy | Rasberry | Blue-violet | Blue-violet |
| to Example 5 | L = 72.1; a = 5.7; | L = 41.3; a = 24.6; | L = 39.2; a = 36.9; | L = 28.1; a = 14.4; | L = 29.8; a = 10.3; |
| | b = 11.2 | b = 0.9 | b = 7.6 | b = −9.6 | b = −7.8 |
| Dye according | Dusky pink | Red | Orange | Red-violet | Dull violet |
| to Example 6 | L = 59.9; a = 21.3; | L = 40.8; a = 40.3; | L = 50.3; a = 43.3; | L = 30.2; a = 31.6; | L = 32.7; a = 23.4; |
| | b = 8.4 | b = 19.2 | b = 41.1 | b = −0.1 | b = −0.8 |
| Dye according | Dusky pink | Dark red | Orange-red | Red-violet | Red-violet |
| to Example 7 | L = 55.1; a = 22.6; | L = 29.9; a = 38.9; | L = 42.9; a = 47.3; | L = 26.0; a = 24.8; | L = 20.6; a = 28.7; |
| | b = 4.0 | b = 16.7 | b = 38.7 | b = −0.1 | b = 1.2 |

### Example 9

### Creamy Oxidative Hair Colorant, Alkaline

| | | |
|---|---|---|
| 15.00 | g (0.53 oz) | cetyl stearyl alcohol (50/50) |
| 5.00 | g (0.18 oz) | glycerin monostearate |
| 2.00 | g (0.07 oz) | Cocamide DEA |
| 10.00 | g (0.35 oz) | sodium lauryl ether sulphate, |
| | | 28% aqueous solution |
| 0.30 | g (0.01 oz) | ascorbic acid |
| 0.40 | g (0.01 oz) | sodium sulfite |
| 0.61 | g (0.02 oz) | dye according to Example 1 |
| 0.32 | g (0.01 oz) | 1,4-diamino-2-methoxymethyl |
| | | benzene sulfate |
| 0.16 | g (0.005 oz) | 4-amino-3-methylphenol |
| 0.50 | g (0.02 oz) | 3-{[phenylmethylidene]amino}phenol |
| | | (CAS [18277-39-9]) |
| 0.13 | g (0.005 oz) | resorcinol |
| 0.17 | g (0.006 oz) | 2,4-dimethyl resorcinol |
| 0.22 | g (0.008 oz) | 2-amino-6-chloro-4-nitrophenol |
| | | hydrochloride |
| 4.50 | g (0.18 oz) | ammonia, 25% aqueous solution |
| balance to 100.00 | g (3.53 oz) | water, de-mineralized |

Immediately before use, 100 grams (3.53 oz) of the herein before color vehicle (pH=10.2) is mixed with 100 grams (3.53 oz) of a 6% aqueous hydrogen peroxide solution, and the ready-to-use oxidative hair colorant obtained is applied to the hereinafter mentioned hair types in the required quantity.

After an action period of 30 minutes at 40° C. (104° F.), the hair is washed with a neutral shampoo, rinsed with water, and dried.

The color results obtained with the various hair types are compiled in Table 2.

TABLE 2

| Hair type | Color | L*a*b* values | | |
|---|---|---|---|---|
| Bleached yak hair | Fiery | Non-colored: | L = 82.8 a = −0.4 | b = 11.6 |
| | red | Colored: | L = 30.6 a = 39.0 | b = 22.2 |
| Human hair that | Copper | Non-colored: | L = 59.1 a = 0.2 | b = 13.1 |
| is 50% gray | red | Colored: | L = 37.6 a = 26.0 | b = 20.4 |
| Human hair with | Brown- | Non-colored: | L = 37.0 a = 5.2 | b = 10.9 |
| shade depth 7/0 | red | Colored: | L = 28.5 b = 16.4 | b = 13.6 |

### Example 10

### Oxidative Hair Colorant, Neutral

| | | |
|---|---|---|
| 4.00 | g (0.14 oz) | Plantaren 2000 UP/NP |
| 0.20 | g (0.007 oz) | Disodium EDTA |
| 5.00 | g (0.18 oz) | ethanol |
| 0.34 | g (0.01 oz) | compound according to Example 7 |
| 0.17 | g (0.006 oz) | 6-hydroxyindole |
| balance to 100.00 | g (3.53 oz) | water, demineralized |

Immediately before use, 100 grams (3.53 oz) of the herein before color vehicle (pH=7.2) is mixed with 100 grams (3.53 oz) of a 6% aqueous hydrogen peroxide solution, and the ready-to-use oxidative hair colorant obtained is applied to bleached hair in the required quantity. After an action period of 30 minutes at 40° C. (104° F.), the hair is washed with a neutral shampoo, rinsed with water, and dried. The hair is colored a cognac shade.

Unless indicated otherwise, all percentages given in the present application represent weight percents.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Pyrazole azomethines of Formula (I), 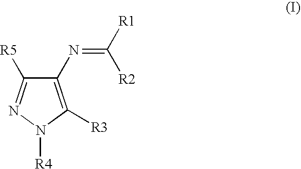
wherein
R1 and R2, independently from one another, represent hydrogen, a dimethylamino group, an aliphatic, aromatic, or heteroaromatic group, which, optionally, is substituted with one or more amino groups, hydroxyl groups, nitrile groups, alkoxy groups, alkylsulfonyl groups, or keto groups, or groups R1 and R2, together with the carbon atom to which they are attached, form cyclic or heterocyclic, saturated or unsaturated compounds, wherein R1 or R2 optionally may additionally contain an ionic grouping;
R3 represents a hydroxyl group, an amino group, a straight-chain or branched C1-C5 alkylamino group, a hydroxyethylamino group, a 2,3-dihydroxy propylamino group, or a benzylamino group;
R4 represents hydrogen, a straight-chain or branched C1-C12 alkyl group, a straight-chain or branched C2-C5 mono- or polyhydroxyalkyl group, a straight-chain or branched C3-C6 alkoxyalkyl group, a phenyl group, or a benzyl group, which, optionally, are substituted with one or more C1-C3 alkyl groups, chlorine atoms, bromine atoms, trifluoromethyl groups, C1-C3 alkoxy groups, hydroxyethoxy groups, nitrile groups, hydroxyl groups, or is a five-membered or six-membered heteroaromatic group; and
R5 represents hydrogen, a straight-chain or branched C1-C6 alkyl group, a C1-C4 hydroxyalkyl group, a C1-C4 aminoalkyl group, a C1-C8 alkylamino group, a di(C1-C8)alkylamino group, a C1-C4 alkylamino(C1-C4)alkyl group, a di(C1-C4)alkylamino(C 1-C4)alkyl group, an aryl group, a heteroaryl group, a carboxylic acid group, a carboxylic acid methylester group, a carboxylic amide group, or a nitrile group;
or R3 and R4 are bridged with one another so that a compound of Formula (Ia) or (Ib) is formed, 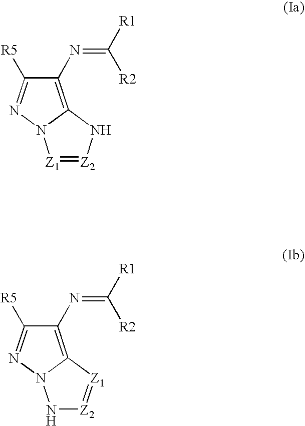
wherein
Z1 and Z2, independently from one another, represent a nitrogen atom or a carbon atom substituted with a hydrogen atom, a straight-chain or branched C1-C5 alkyl group, a benzyl residue, a trifluoromethyl group, a hydroxymethyl group, a hydroxyethyl group, a methoxy group, or a phenyl group substituted with methyl group, a chlorine atom, a hydroxy group, a methoxy group, or a trifluoromethyl group, and residue groups R1, R2, and R5 have the aforementioned meaning.

2. Pyrazole azomethine as recited in claim 1, wherein it is selected from the group consisting of 1-methyl-N⁴-[phenylmethylidene]-1H-pyrazole-4,5-diamine, N⁴-{[4-(methyloxy)phenyl]methylidene}-1-pentyl-1H-pyrazole-4,5-diamine, 4-({[5-amino-1-(1-methylethyl)-1H-pyrazol-4-yl]imino}methyl)-2-(methyloxy)phenol, 1-[2-(dimethylamino)ethyl]-N⁴-[(4-methoxyphenyl)methylidene]-1H-pyrazole-4,5-diamine, 3-({[5amino-1-(2-hydroxyethyl)-1H-pyrazol-4-yl]imino}methyl)-1-methyl pyridinium methylsulphate, 4-({[5-amino-1-(4-methoxyphenyl)-1H-pyrazol-4-yl]imino}methyl)-2-methoxyphenol, 1-(2-pyridinyl)-N⁴-[3-pyridinyl methylidene]-1H-pyrazole-4,5-diamine, 4-({[5-amino-1-(4-methylbenzyl)-1H-pyrazol-4-yl]imino}methyl)phenol, and 3-methyl-N4-{[4-(methyloxy)phenyl]methylidene}-1-phenyl-1H-pyrazole-4,5-diamine, or mixtures of these compounds.

3. An agent for oxidative coloring of keratin fibers, wherein said agent contains at least one pyrazole azomethine as recited in claim 1.

4. The agent for oxidative coloring of keratin fibers as recited in claim 3, wherein the compound of Formula (I) is present in a total quantity of from about 0.005 to about 20 percent by weight.

5. The agent for oxidative coloring of keratin fibers as recited in claim 3, wherein said agent also contains at least one coupler substance, at least one developer substance, or mixtures thereof.

6. The agent for oxidative coloring of keratin fibers as recited in claim 3, wherein said agent also contains at least one natural, nature-identical, or synthetic direct-penetrating dye.

7. The agent for oxidative coloring of keratin fibers as recited in claim 3, wherein said agent exhibits a pH value of from about 6 to about 11.5.

8. The agent for oxidative coloring of keratin fibers as recited in claim 3, wherein said agent is mixed with an oxidizing agent before use.

9. The agent for oxidative coloring of keratin fibers as recited in claim 3, wherein said agent is a hair colorant.
